SEQUENCE LISTING (24 sequences)

SEQ ID NO: 1 - protein, 4 aa, Artificial Sequence
  MOD_RES (4): AMIDATION
RGFF

SEQ ID NO: 2 - protein, 4 aa, Artificial Sequence
  MOD_RES (4): residue is Cyclohexylalanine
RGFX

SEQ ID NO: 3 - protein, 4 aa, Artificial Sequence
  SITE (2): residue is D-Ala
RXFF

SEQ ID NO: 4 - protein, 4 aa, Artificial Sequence
  MOD_RES (4): AMIDATION
RLFF

SEQ ID NO: 5 - protein, 6 aa, Artificial Sequence
  MOD_RES (6): AMIDATION
RLFDNA

SEQ ID NO: 6 - protein, 6 aa, Artificial Sequence
  SITE (4): residue is aminosuccinimide
RLFXNA

SEQ ID NO: 7 - protein, 8 aa, Artificial Sequence
  SITE (6): residue is aminosuccinimide
LSRLFXNA

SEQ ID NO: 8 - protein, 8 aa, Artificial Sequence
  SITE (6): residue is aminosuccinimide
LSKLFXNA

SEQ ID NO: 9 - protein, 8 aa, Artificial Sequence
  SITE (6): residue is aminosuccinimide
LSGLFXNA

SEQ ID NO: 10 - protein, 8 aa, Artificial Sequence
  SITE (6): residue is aminosuccinimide
LSRLYXNA

SEQ ID NO: 11 - protein, 8 aa, Artificial Sequence
  SITE (6): residue is aminosuccinimide
LSRLGXNA

SEQ ID NO: 12 - protein, 4 aa, Artificial Sequence
  MOD_RES (4): AMIDATION
AGFF

SEQ ID NO: 13 - protein, 4 aa, Artificial Sequence
  MOD_RES (4): AMIDATION
RAFF

SEQ ID NO: 14 - protein, 4 aa, Artificial Sequence
  MOD_RES (4): AMIDATION
RGAF

SEQ ID NO: 15 - protein, 4 aa, Artificial Sequence
  MOD_RES (4): AMIDATION
RGFA

SEQ ID NO: 16 - protein, 4 aa, Artificial Sequence
  MOD_RES (4): AMIDATION
KGFF

SEQ ID NO: 17 - protein, 4 aa, Artificial Sequence
  MOD_RES (1): Orn
XGFF

SEQ ID NO: 18 - protein, 4 aa, Artificial Sequence
  SITE (2): residue is beta-Alanine
RXFF

SEQ ID NO: 19 - protein, 4 aa, Artificial Sequence
  MOD_RES (4): AMIDATION
RGYF

SEQ ID NO: 20 - protein, 4 aa, Artificial Sequence
  SITE (3): residue is beta-Cyclohexyl-L-alanine
RGXF

SEQ ID NO: 21 - protein, 4 aa, Artificial Sequence
  MOD_RES (4): AMIDATION
RGFY

SEQ ID NO: 22 - protein, 4 aa, Artificial Sequence
  SITE (2): residue is D-alanine
RXFY

SEQ ID NO: 23 - protein, 4 aa, Artificial Sequence
  SITE (2): residue is D-alanine
RXFY

SEQ ID NO: 24 - protein, 4 aa, Artificial Sequence
  SITE (2): residue is D-alanine
RXYF

(19) United States
(12) Patent Application Publication
(10) Pub. No.: US 2004/0054130 A1
(43) Pub. Date: Mar. 18, 2004

(54) Insulin potentiating peptides

(76) Inventors: Frank Man-Woon Ng, Kew (AU); Woei-Ji Jiang, Glen Waverley (AU)

Correspondence Address: KLARQUIST SPARKMAN, LLP, 121 SW SALMON STREET, SUITE 1600, PORTLAND, OR, 97204, US

(21) Appl. No.: 10/240,578

(22) PCT Filed: Mar. 30, 2001
(86) PCT No.: PCT/AU01/00354

(30) Foreign Application Priority Data
Mar. 31, 2000 (AU) PQ 6618

(51) Int. Cl. A61K 38/06; C07K 5/06

(52) U.S. Cl. 530/330; 514/18

(57) ABSTRACT

This invention relates to compounds which have the ability to potentiate the physiological activity of insulin, and in particular to small peptide compounds. The compounds are useful in the treatment of conditions related to insulin resistance, such as non-insulin dependent diabetes mellitus (NIDDM) and obesity. The invention provides a peptide or peptidomimetic compound which has the ability to potentiate one or more of the physiological activities of insulin, in which the peptide comprises the sequence: W-X-Y-Z where W is a basic amino acid, such as lysine, arginine, homolysine, homoarginine or ornithine; X is a neutral aliphatic amino acid, in either the L- or the D-form, such as glycine, leucine, alanine, β-alanine or isoleucine, homoleucine, norleucine, homonorleucine, cyclohexylalanine, or homocyclohexylalanine; Y is an aromatic amino acid, such as phenylalanine or tyrosine; and Z is an amino acid or amino acid analogue which has a side chain having π or delocalised electrons, with the proviso that the peptide is not Arg-Gly-Phe-Phe, Arg-Gly-Ser-Arg-Leu-Phe-Phe-Asn-Tyr-Ala-Leu-Val, Arg-Leu-Phe-Asu-Asn-Ala, or Leu-Ser-Arg-Leu-Phe-Asu-Asn-Ala. Compositions and methods of treatment are also within the scope of the invention.

This invention relates to compounds which have the ability to potentiate the physiological activity of insulin, and in particular to small peptide compounds. The compounds are useful in the treatment of conditions related to insulin resistance, such as non-insulin dependent diabetes mellitus (NIDDM) and obesity.

### BACKGROUND OF THE INVENTION

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art, in Australia or in any other country.

Insulin resistance is a physiological state in which insulin induces a diminished response from target tissues. This resistance to insulin action is a major pathogenic factor associated with non-insulin-dependent diabetes mellitus (NIDDM) (Keen, 1994), obesity (Felber et al, 1993; Truglia et al, 1985), hypertension (Baba and Neugebauer, 1994), and coronary heart disease (CHD) (Zavaroni et al, 1989).

Type II diabetes (non-insulin dependent diabetes) is characterised by inadequate control over blood sugars with an elevated level of plasma insulin. The biochemical causes are known to vary between individuals, although a common element in the development of an insensitivity is the deficiency of the target organs to respond to plasma insulin. Subsequently the pancreas has increasing difficulty supplying the increasing amount of insulin required to achieve the optimal blood glucose levels, particularly after meals. The insulin-producing islet cells of the pancreas ultimately suffer from excessive use and begin to fail, further limiting the amount of insulin which can be produced. At this stage the patient may become overtly type I diabetic, requiring insulin doses to maintain blood glucose.

Risk factors for type II diabetes include old age, obesity and inherited genetic factors. There does not appear to be a dominant biochemical defect which causes the underlying insulin insensitivity. In principle, insulin insensitivity may be caused by interference with insulin before binding with the insulin receptor, receptor defects, defects at any of many possible points in the intracellular signalling pathways, defects in the glucose transport channels which insulin upregulates, or any combination of these factors.

The standard initial step in therapy is modification of diet and lifestyle. If this fails, a range of pharmaceutical agents is available for treating the condition, such as sulphonylureas, biguanides and thiazolidinediones. Perhaps because the disease has no common biochemical cause, responses to the drugs differ between individuals, and the drugs have significant side-effects.

The insulin-potentiating effects of certain synthetic peptide amides corresponding to the C-terminal fragment of the B-chain of insulin have been demonstrated by ourselves and others (Ng et al, 1989; Weitzel et al, 1971). The insulin B-chain(INSB) fragment from amino acid residues 22-25, Arg-Gly-Phe-Phe, has been shown to be involved in binding of the insulin molecule to its receptor (Pullen et al, 1976). This fragment is referred to herein as INSB(22-25). De Meyts et al reported that the INSB(22-25) fragment interacted with the residues 83-94, Arg-Gly-Ser-Arg-Leu-Phe-Phe-Asn-Tyr-Ala-Leu-Val, of the α-subunit of the insulin receptor (De Meyts et al, 1990). The remarkable resemblance between these sequences in insulin and its receptor apparently facilitates insulin-receptor binding by means of a Phe^{B25}Phe⁸⁹ interaction, which is similar to the Phe^{B25} Phe^{B25} interaction in insulin dimerization.

In the early 1980s, similar insulin-potentiating effects were also shown both in vitro and in vivo with peptide amides from the amino-terminus of human growth hormone (hGH) (Ng et al, 1980). It was found that the peptide required an α-aminosuccinimide (Asu) modification in the residue Asp¹¹ for biological activity (Robson et al, 1990). Asu¹¹-hGH(6-13) peptide, Leu-Ser-Arg-Leu-Phe-Asu-Asn-Ala, was shown to improve glucose tolerance in the insulin-resistant Zucker fatty (fa/fa) rats, as demonstrated by the glucose clamp technique (Lim et al, 1995). The amino acid sequence 8-11 of hGH, Arg-Leu-Phe-Asu-Asn-Ala, incorporating the Asn modification, elicits an insulin-potentiating effect. The four resides at the amino terminus of this peptide appear to be homologous to the corresponding sequence of the insulin tetrapeptide INSB(22-25). Conformational analysis of this peptide using NMR and molecular modelling suggested that a structural constraint, a Type II′β turn, was introduced by Asu (Ede et al, 1994).

It is known that peptides containing the minimal sequence hGH(6-13) are hypoglycaemic, and this sequence appears to account for the hypoglycaemic actions of intact hGH(1-191). The in vitro effects of hGH(6-13) include:

(a) facilitation of insulin binding to membrane receptors;

(b) acceleration of glucose transport in isolated cells;

(c) activation of intracellular enzymes for glucose and glycogen metabolism;

(d) augmentation of glucose oxidation in muscle, adipose tissue and liver; and

(e) enhancement of glucose-induced release of insulin from pancreatic islets.

The in vivo effects of hGH(6-13) include an increase of glucose disposal in glucose tolerance tests without causing excessive hypoglycaemia, and enhanced tissue sensitivity to the action of insulin.

The similar insulin-potentiating actions of peptide fragments from insulin, insulin receptor, and hGH may be due to a common functional motif. The present study was therefore undertaken in order to identify the insulin-potentiating motif, based on the sequence structures of insulin, insulin receptor and hGH, with the objective of developing novel drugs in the treatment of NIDDM and their effects-on obesity.

Insulin-potentiating effects were demonstrated both in vitro and in vivo with a series of peptide amide analogues corresponding to the amino acid sequence 22-25 of the B-chain of insulin, residues 86-89 of the α-subunit of insulin receptor, and the N-terminal region of human growth hormone. Structure-function studies suggest that the biological action may be intrinsic to a four-residue motif with a basic amino acid in position 1, a neutral aliphatic amino acid in position 2, an aromatic amino acid in position 3, and an amino acid with a side-chain having π or non-binding electrons in position 4. This molecular motif provides a new direction for the construction of novel therapeutic agents for the treatment of insulin-resistance related diseases such as non-insulin dependent diabetes mellitus (NIDDM) or obesity.

### SUMMARY OF THE INVENTION

According to a first aspect, the invention provides a peptide which has the ability to potentiate one or more of the physiological activities of insulin, in which the peptide comprises the sequence:

W-X-Y-Z

where W is a basic amino acid, such as lysine, arginine, homolysine, homoarginine or ornithine;

X is a neutral aliphatic amino acid, in either the L- or the D-form, such as glycine, leucine, alanine, β-alanine or isoleucine, homoleucine, norleucine, homonorleucine, cyclohexylalanine, or homocyclohexylalanine;

Y is an aromatic amino acid, such as phenylalanine or tyrosine; and

Z is an amino acid or amino acid analogue which has a side chain having π or delocalised electrons,

with the proviso that the peptide is not Arg-Gly-Phe-Phe, Arg-Gly-Ser-Arg-Leu-Phe-Phe-Asn-Tyr-Ala-Leu-Val, Arg-Leu-Phe-Asu-Asn-Ala, or Leu-Ser-Arg-Leu-Phe-Asu-Asn-Ala.

Preferably the amino acid or amino acid analogue Z is one with a cyclic side chain, such as phenylalanine, tyrosine, tryptophan, α-amino succinimide, homophenylalanine or histidine.

It will be clearly understood that the sequence W-X-Y-Z is a minimum sequence, and may be extended at either the N- or C-terminal, provided that the ability to potentiate insulin activity is retained.

While the invention has been primarily exemplified in relation to peptides, it will also be understood that the peptide linkage between the residues may be replaced by a non-peptide bond provided that the ability to potentiate insulin activity is retained. The person skilled in the art will be aware of suitable such modifications.

Sequences encompassing conservative substitutions of amino acids are also within the scope of the invention, provided that the biological activity is retained.

It is to be clearly understood that the compounds of the invention include peptide amides and non-amides, and peptide analogues, including but not limited to the following:

1. Compounds in which one or more amino acids is replaced by its corresponding D-amino acid. The skilled person will be aware that retro-inverso amino acid sequences can be synthesised by standard methods; see for example Chorev and Goodman, 1993;

2. Peptidomimetic compounds, in which the peptide bond is replaced by a structure more resistant to metabolic degradation. See for example Olson et al, 1993; and

3. Compounds in which individual amino acids are replaced by analogous structures for example, gem-diaminoalkyl groups or alkylmalonyl groups, with or without modified termini or alkyl, acyl or amine substitutions to modify their charge.

The use of such alternative structures can provide significantly longer half-life in the body, since they are more resistant to breakdown under physiological conditions, or to improve bioavailability.

Methods for combinatorial synthesis of peptide analogues and for screening of peptides and peptide analogues are well known in the art (see for example Gallop et al, 1994; Hogan, 1997). It is particularly contemplated that the compounds of the invention are useful as templates for design and synthesis of compounds of improved activity, stability and bioavailability. Mimetics of amino acid side chains are known in the art. For example, mimetics of arginine side chains are disclosed in PCT/AU98/00490 (WO 99/00406) by The University of Queensland.

In a preferred embodiment of the invention, the peptide is selected from the group consisting of:

| | |
|---|---|
| Arg-D-Ala-Phe-Phe, | (SEQ ID NO. 3) |
| Arg-Leu-Phe-Phe, | (SEQ ID NO. 4) |
| Arg-Leu-Phe-Asu-Asn-Ala, | (SEQ ID NO. 6) |
| Leu-Ser-Arg-Leu-Tyr-Asu-Asn-Ala, | (SEQ ID NO. 7) |
| Leu-Ser-Lys-Leu-Phe--Asu-Asn-Ala, | (SEQ ID NO. 8) |
| Leu-Ser-Arg-Leu-Tyr-Asu-Asn-Ala, | (SEQ ID NO. 10) |
| Arg-β-A1a-Phe-Phe, | (SEQ ID NO. 18) |
| Arg-Gly-Tyr-Phe, | (SEQ ID NO. 19) |
| Arg-D-Ala-Phe-Tyr, | (SEQ ID NO. 22) |
| Arg-D-Ala-Phe-Tyr-me, and | (SEQ ID NO. 23) |
| Arg-D-Ala-Tyr-Phe, | (SEQ ID NO. 24) |

More preferably the peptide is Arg-D-Ala-Phe-Phe-NH₂ (SEQ ID NO. 3)or Arg-D-Ala-Tyr-Phe-NH₂ (SEQ ID NO. 24).

In a second aspect, the invention provides a composition comprising a peptide according to the invention, together with a pharmaceutically-acceptable carrier.

Methods and pharmaceutical carriers for preparation of pharmaceutical compositions are well known in the art, as set out in textbooks such as Remington's Pharmaceutical Sciences, 17th Edition, Mack Publishing Company, Easton, Pa., USA., and may be selected according to the desired route of administration.

In a third aspect, the invention provides a method of treatment of a pathological condition associated with insulin resistance, comprising the step of administering an effective amount of a peptide according to the invention to a subject in need of such treatment. Preferably the condition is non-insulin dependent diabetes mellitus or obesity. More preferably the condition is non-insulin-dependent diabetes mellitus.

In a fourth aspect, the invention provides a method of treatment of a pathological condition associated with insulin resistance, comprising the step of administering an effective amount of a compound which mimics the action of the binding region of INSB 22:25 on the insulin receptor to a subject in need of such treatment.

The dose and route of administration will depend on the nature of the condition to be treated, and the condition, previous treatment and general state of health of the subject to be treated, and will be at the discretion of the attending physician. However, in general it is contemplated that the dose will be in the range 0.1 to 100 mg/kg body weight, preferably 1 to 50 mg/kg body weight, more preferably 1 to 10 mg/kg body weight.

Although any desired route of administration may be used, including both enteral and parenteral routes such as oral administration or subcutaneous or intramuscular injection, preferably the peptide is administered orally or sublingually. One or more doses per day may be administered, preferably at meal times so as to reduce the peak post-prandial blood glucose level.

While the biological activity is demonstrated herein by measuring in vitro and in vivo insulin-potentiating effects, it will be clearly understood that primary screening of putative insulin-potentiating peptides may be achieved by any convenient method, preferably a high-throughput method of measuring binding to insulin receptor, using biosensor assays. Suitable methods are well known in the art. It will be also understood that putative peptides and peptidometic compounds may readily be synthesised using automated high-throughput solid phrase peptide synthesis.

For the purposes of this specification it will be clearly understood that the word “comprising” means “including but not limited to”, and that the word “comprises” has a corresponding meaning.

### BRIEF DESCRIPTION OF THE FIGURES

FIG. 1 shows the sensitivity of hemidiaphragm muscle tissue to the effect of insulin on glucose incorporation into glycogen. Mean±SEM; data from 8 animals.

FIG. 2 shows the effects of peptide 1 (Δ), peptide 2 (⊖), peptide 3 (▾), peptide 4 (▴), peptide 5 (□) and peptide 6 (●) (panel A) and peptide 7 (◯) and peptide 8 (●), peptide 9 (□), peptide 10 (Δ), peptide 11 (▴) (panel B) on the rate of glucose incorporation into glycogen in hemidiaphragm tissue by increasing concentrations of peptides, together with exogenous insulin (1 mU/ml). Tissues from the same rat were used for all groups. Mean±SEM; data from 8 animals.

FIG. 3 shows the effect of peptide 1 (Δ), peptide 2 (&Rectversolid;), peptide 3 (▾), peptide 4 (▴), peptide 5 (□), and peptide 6 (●) on blood glucose levels of Zucker rats. Animals were given i.p. saline or peptide (10 μmol/kg body weight), and the reductions of blood glucose were determined. Basal blood glucose level of all animals were 6.2±0.5 mmol/L before experimentation. * denotes that differences between the peptide treated and buffer control groups (◯) are statistically significant (p<0.05) at the indicated time.

### METHODS

Animals

Zucker fatty (fa/fa) female rats (440-470 g) of 30 weeks old and normal Wistar male rats (140-160 g) of 5 weeks old were used. The animals were fed ad libitum on rat pellets (Clark King, Melbourne, Australia) with free access to water at all times, and housed in the departmental animal house.

Peptide Synthesis

The peptide amide analogues discussed in Examples 1-6 were prepared by manual solid-phase synthesis, using the Fmoc strategy and Rink amide resin. The in situ coupling reaction was performed with diisopropylcarbodiimide (DIC)/1-hydroxybenzotriazol (HOBt). After synthesis, the peptide was cleaved from the resin and side-chain protective groups were removed by treatment with Reagent K (King et al, 1990) for 1.5 hr, either at room temperature for peptides 1-4 or at 4° C. for peptides 5-11. Peptides were purified by reverse phase high performance liquid chromatography (RP-HPLC) using a preparative C18-column (21.2 mm×25 cm, Supelco) and an acetonitrile gradient (0-50% in 50 min). The purity of peptides was at least 99%. The amino acid composition and the molecular weight determinations were determined either using a Waters Pico Tag system or by fast atom bombardment-mass spectrometry (FAB—MS).

In vitro Measurements of Glycogen Synthesis in Muscle

In vitro insulin-potentiating effects of the synthetic peptide analogues were assessed by measuring the rates of exogenous glucose incorporation into glycogen in rat hemidiaphragms (Lim et al, 1992). Hemidiaphragms from overnight-fasted Zucker fatty(fa/fa) female rats were dissected, and divided into segments of approximate 35-50 mg each. Tissues from the same rat were used for all groups. The tissue was incubated in 2 ml of Krebs-Ringer bicarbonate (KRB) buffer (pH 7.4) containing [¹⁴C]glucose (5.5 mM, final specific activity 0.05 mCi/mmol) under an atmosphere of 95% 0₂-5% CO₂ at 37° C. for 1.5 hr. After incubation, tissues were removed, washed with cold buffer and blotted. Tissues were digested, the muscle glycogen was precipitated and the ¹⁴C-radioactivity was counted in a Wallac 1410 liquid scintillation counter. The biological activity of peptide analogues was measured as the rate of mmol glucose incorporation into muscle glycogen/g tissue/hr.

The sensitivity of hemidiaphragm muscle tissue to insulin (0.1-100 mU/ml) on glycogen synthesis was first analyzed. The dose response curves for peptide analogues on the insulin-potentiating effect to glycogen synthesis were then measured using cumulative increasing concentrations (10^{-3 10} μmol/ml) of peptides in the presence of insulin (1 mU/ml). The biological activity of each peptide analogue was measured as the rate of glucose incorporation into muscle glycogen (μmol/g tissue/hr), and represented by the mean±SEM from eight determinations.

Basal Blood Glucose Determination

Overnight-fasted Zucker fatty(fa/fa) female rats were anaesthetized with sodium pentobarbitone (60 mg/kg body weight). After 45 min, basal blood glucose samples were taken from the tail vein, followed immediately by intraperitoneal (i.p.) injection of saline (control) or the peptide analogue (test, 10 μmol/kg body weight) in 0.4 ml of saline. Blood samples were taken at 15, 30, 60, 90, 120, 150 minutes after injection, and the blood glucose level in each sample was measured immediately by the glucose oxidase method, using a YSI Model-2300 STAT glucose analyzer (Yellow Spring, Ohio). Six animals for each group were used.

Intravenous Insulin Tolerance Test (IVITT)

IVITTs (0.1 U insulin/kg body weight) were performed on overnight-fasted Wistar male rats as previously described (Lim et al, 1992). Blood samples were taken for glucose estimation at 15, 30, 45, 60 min after the commencement of the tests. Six animals in each group were used.

Statistical Analysis

The Student's t-test was used to analyze the results. P values of <0.05 were accepted as statistically significant.

### EXAMPLE 1

### Aminosuccinimide Modification of hGH Peptides

α-aminosuccinimide derivatives of hGH peptides were prepared by a two-step approach, in which the aspartyl¹¹ β-methyl ester of hGH peptides is subjected to subsequent displacement of the ester group by the neighbouring amide nitrogen of Asn¹², resulting in formation of an α-aminosuccinimide derivative.

hGH peptides with an α-aminosuccinimide(Asu) modification in the aspartyl residue were prepared by methyl esterification of the β-carboxylic group of Asp¹¹, followed by base-catalyzed de-esterification and ring closure according to the procedure of Stephenson et al (Stephenson and Clarke, 1989). Peptide (80 μmol) was first esterified by 30 ml of 0.08 N hydrochloric acid (HCl) in methanol at 20° C. overnight. Purified peptide ester (50 μmol) was incubated in 100 mL of 0.2 M sodium phosphate buffer (pH 7.4) at 20° C. or 37° C. Asu formation was monitored with RP-HPLC using an analytical C18-column (4.6 mm×25 cm, Vydac) at 214 nm. The reaction was terminated by adding diethyl ether and the Asu-peptides were purified by RP-HPLC. The peptides synthesised for this study are summarised in Table 1. Peptide 3 was subsequently designated compound ADD9903.

TABLE 1
Sequences of Synthetic Peptides

| | | |
|---|---|---|
| Peptide | Sequence | Peptide Amide Analogues |

| | | | | |
|---|---|---|---|---|
| 1 | INSB | (22-25) | Arg-Gly-Phe-Phe | (SEQ ID NO. 1) |
| 2 | Cha²⁵-INSB | (22-25) | Arg-Gly-Phe-Cha | (SEQ ID NO. 2) |
| 3 | D-Ala²³-INSB | (22-25) | Arg-D-Ala-Phe-Phe | (SEQ ID NO. 3) |
| 4 | INSREC | (86-89) | Arg-Leu-Phe-Phe | (SEQ ID NO. 4) |
| 5 | hGH | (8-13) | Arg-Leu-Phe-Asp-Asn-Ala | (SEQ ID NO. 5) |
| 6 | AsU¹¹-hGH | (8-13) | Arg-Leu-Phe-Asu-Asn-Ala | (SEQ ID NO. 6) |
| 7 | Asu¹¹-hGH | (6-13) | Leu-Ser-Arg-Leu-Phe-Asu-Asn-Ala | (SEQ ID NO. 7) |
| 8 | Lys⁸, Asu¹¹-hGH | (6-13) | Leu-Ser-Lys-Leu-Phe-Asu-Asn-Ala | (SEQ ID NO. 8) |
| 9 | Gly⁸, Asu¹¹-hGU | (6-13) | Leu-Ser-Gly-Leu-Phe-Asu-Asn-Ala | (SEQ ID NO. 9) |
| 10 | Tyr¹⁰, Asu¹¹-hGH | (6-13) | Leu-Ser-Arg-Leu-Tyr-Asu-Asn-Ala | (SEQ ID NO. 10) |
| 11 | Gly¹⁰, Asu¹¹-hGH | (6-13) | Leu-Ser-Arg-Leu-Gly-Asu-Asn-Ala | (SEQ ID NO. 11) |

INSB: insulin B-chain
INSREC: α-subunit of the insulin receptor
hGH: human growth hormone
Asu: aminosuccinimide
Cha: β-cyclohexyl-L-alanine

Samples wereanalyzed by RP-HPLC using a linear gradient of acetonitrile from 0%-40% over 40 min. Peak areas beneath the identified peaks were regarded as the molar quantities.

For example, 55% conversion of the hGH(8-13) peptide to the α-aminosuccinimide form could be achieved in 2.5 hr at 20° C., as shown in Table 2.

TABLE 2
Aminosuccinimide Modification of hGH (8-13) Peptide

| Temperature | | | | | | |
|---|---|---|---|---|---|---|
| (° C.) | 20 | | | 37 | | |
| Time | Composition | | | Composition | | |
| (min) | E | I | A | E | I | A |
| 0 | 100 | 0 | 0 | 100 | 0 | 0 |
| 3 | 96 | 4 | 0 | 92 | 8 | 0 |
| 15 | 89 | 11 | 0 | 27 | 52 | 21 |
| 30 | 81 | 19 | 0 | 17 | 44 | 39 |
| 60 | 67 | 30 | 4 | 3 | 17 | 80 |
| 150 | 32 | 55 | 13 | <1 | 1 | >98 |
| 240 | 29 | 49 | 22 | <1 | <1 | >99 |

E = aspartyl¹¹ β-methyl ester of hGH (8-13),
I = Asu¹¹-hGH (8-13),
A = hGH (8-13) and other isomers.

However, aspartyl isomers of hGH(8-13) peptide were formed when the reaction was carried out either for a longer period or at higher temperature, due to the descomposition of the succinimide structure. Total yields of Asu¹¹-hGH peptide analogues were 35%-50%, as calculated from the initial Rink resin loading.

### EXAMPLE 2

### Insulin-Potentiating Effects on Glycogen Synthesis

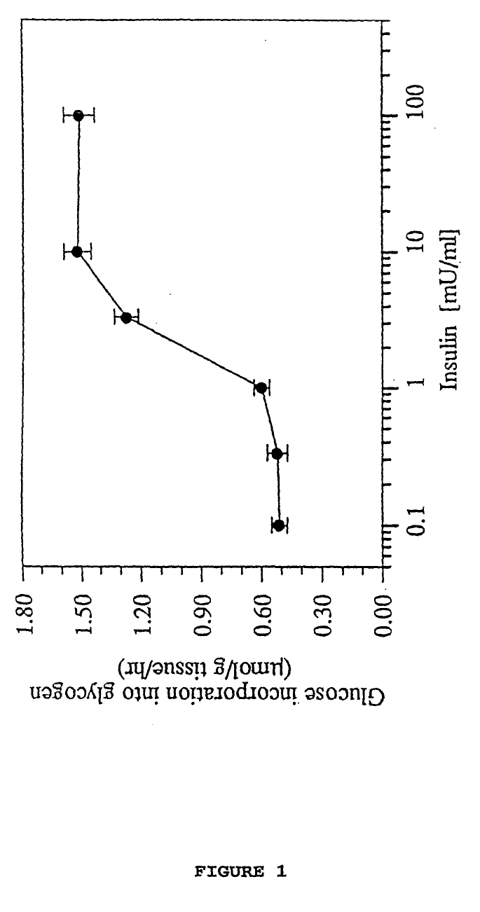

To determine the insulin-potentiating effect of the peptide analogues, the rates of incorporation of glucose into muscle glycogen were measured. The rates of glycogen synthesis (μmol/g tissue/hr) were 0.52±0.05, 0.60±0.04, 1.27±0.06 and 1.52±0.07 inresponse to 0.33, 1, 3.33 and 10 mU/ml insulin respectively, as shown in FIG. 1. This indicated that the stimulation of glycogen production was markedly accelerated when the amount of insulin was greater than 1 mU/ml.

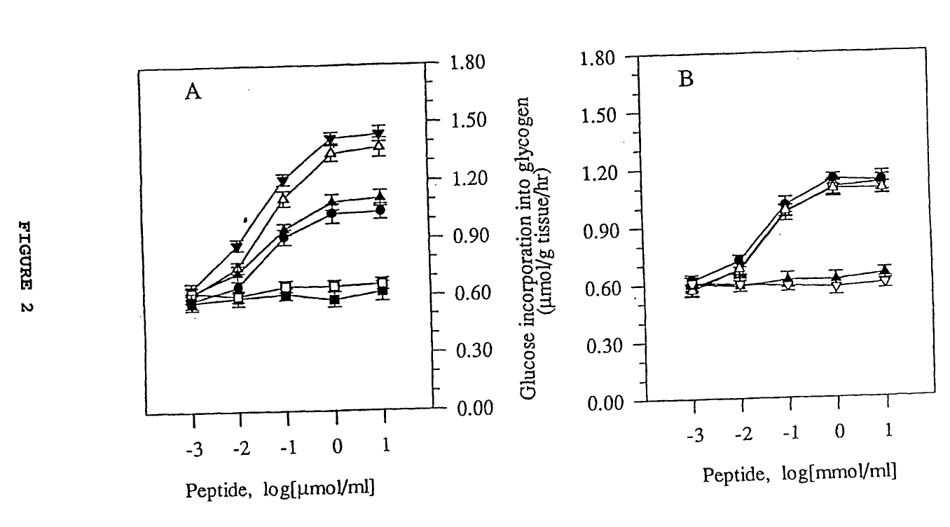

The insulin-potentiating effect of the peptide analogues was then observed by studying their dose response. curves for glucose incorporation into glycogen in the presence of 1 mU/ml exogenous insulin. The effects of peptides 1, 3, 4, 6, 7, 8 and 10 were evident at doses higher than 0.01 μmol/ml, and continued to increase with increasing peptide concentration to 1 μmol/ml, as shown in FIGS. 2A and 2B. The maximum stimulation for the rate of glycogen synthesis, up to 1.44±0.04 (mol/g tissue/hr), was observed in response to 10 μmol/ml of Arg-D-Ala-Phe-Phe amide (Peptide 3). However, insulin-potentiating activity was abolished if either the Arg or the Phe residue of INSB(22-25) was replaced by Gly or β-cyclohexyl-L-alanine (Cha) respective, and if hGH(8-13) did not have the Asn modification.

### EXAMPLE 3

### Hypoglycaemic Effect of INSB(22-25), INSREC(86-89) and Asu¹¹-hGH(8-13) Peptides in Zucker Fatty (fa/fa) Rats

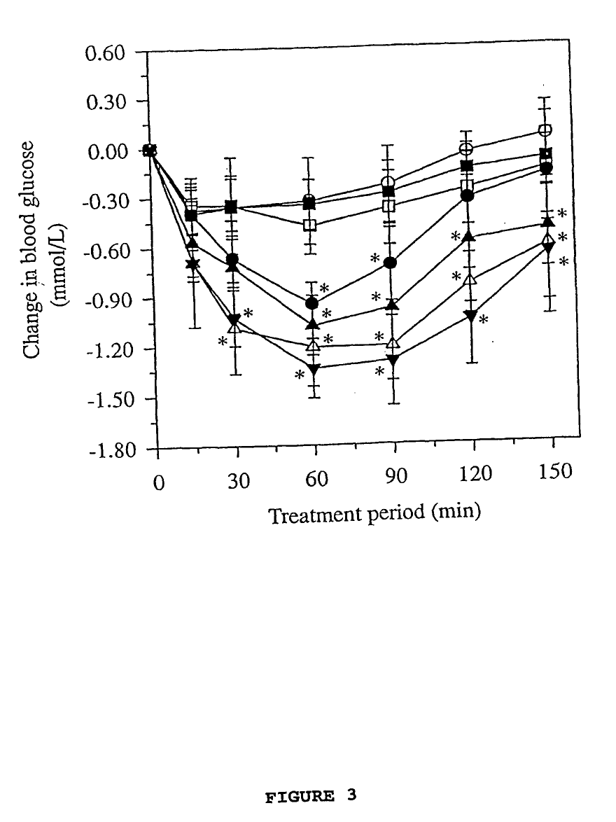

The insulin-potentiating effects of the peptide analogues were demonstrated using insulin-resistant Zucker fatty (fa/fa) rats. The reduction of basal blood glucose levels in animals by different peptide analogues administered intraperitoneally (i.p.)at a dose of 10 μmol/kg body weight was measured for over 150 min. The results are shown in FIG. 3.

Peptides 1, 2, 3 and 6 showed significant hypoglycaemic effects (p<0.005) during 60-90 min after administration, as compared with the control animals which were given an identical volume of saline. The potency of the peptide analogues decreased in the following order: Arg-D-Ala-Phe-Phe>Arg-Gly-Phe-Phe>Arg-Leu-Phe-Phe>Arg-Leu-Phe-Asu-Asn-Ala.

The Arg-Gly-Phe-Cha and hGH(8-13) peptide amide analogues showed no hypoglycaemic effect.

### EXAMPLE 4

### Structure-Function Study of hGH Peptide Analogues

IVITTs were performed on normal male Wistar rats after a single intravenous (i.v.) injection of the hGH peptide analogues at a dose of 5 μmol/kg body weight. The insulin-potentiating effects of peptides 6, 7, 8 and 10 on decreasing blood glucose levels of treated animals became significant since 30 min after the commencement of the test. Bioactivity was retained when the Arg⁸ or Phe¹⁰ residue of Asu¹¹-hGH(6-13) peptide was substituted with Lys or Tyr respectively (1.92±0.17 or 1.62±0.18 vs. 1.65±0.12 mmol/L at 45 min), as shown in Table 3. However, no insulin-potentiating effect was observed when either Arg⁸ or Phe¹⁰ was substituted by Gly (0.92±0.08 or 1.08±0.08 mmol/L respectively vs. 1.00±0.10 mmol/L of control at 45 min). Asu¹¹-hGH(8-13) also elicited this insulin-potentiating effect, but with lower potency. Its linear analogue, Asn¹¹-hGH(8-13), showed no such effect.

TABLE 3
Potentiating Effect of hGH Peptide Analogues
(5 μmol/kg body weight) on Intravenous Insulin Tolerance
Tests (IVITTs).

Time
(min)

| | | | |
|---|---|---|---|
| 15 | 30 | 45 | 60 |

| | |
|---|---|
| Peptide | Reduction in Blood Glucose (mmol/L) |

| | | | | |
|---|---|---|---|---|
| 5 | 0.90 ± 0.09 | 1.01 ± 0.11 | 1.07 ± 0.15 | 1.06 ± 0.12 |
| 6 | 0.93 ± 0.17 | 1.58 ± 0.12* | 1.50 ± 0.15* | 1.50 ± 0.19* |
| 7 | 1.08 ± 0.09 | 1.38 ± 0.13* | 1.65 ± 0.12* | 1.77 ± 0.19* |
| 8 | 1.08 ± 0.10 | 1.35 ± 0.14* | 1.92 ± 0.17* | 1.78 ± 0.17* |
| 9 | 0.87 ± 0.12 | 0.80 ± 0.14 | 0.88 ± 0.15 | 0.74 ± 0.16 |
| 10 | 1.03 ± 0.10 | 1.52 ± 0.12* | 1.62 ± 0.18* | 1.60 ± 0.34* |
| 11 | 0.68 ± 0.05 | 0.96 ± 0.06 | 1.08 ± 0.08 | 1.12 ± 0.06 |
| Control | 0.86 ± 0.10 | 0.92 ± 0.14 | 1.00 ± 0.10 | 0.98 ± 0.10 |
| (without | | | | |
| peptide) | | | | |

All data represent the Mean ± SEM for 6 animals in each group.
*denotes that differences between the peptide treated and control group are statistically significant (p < 0.05) at the indicated time. Basal blood glucose level of all animals was 3.4 ± 0.4 mmol/L before experimentation.

### EXAMPLE 5

### Effect of Acute Oral Administration

Overnight fasted Zucker fatty (fa/fa) female rats were administered peptide 3 (ADD9903) by oral gavage at a concentration of 20 μmol/kg of body weight. Rats were then immediately anaesthetized with nembutal administered intraperitoneally in order to avoid variations arising due to activity of the rats. Blood samples were collected from the tail vein at time 0 min (immediately after oral gavage and before anaesthetic), 60 min, 120 min and 180 min, and analyzed for blood glucose by the glucose oxidation method using a YSI Model-2300 STAT glucose analyzer (Yellow Spring, Ohio). Six rats were analyzed for each of the control and treated groups.

The oral administration of peptide 3 to female Zucker (fa/fa) rats significantly reduced blood glucose levels compared to control rats. A significant reduction in blood glucose was observed 60 min after peptide 3 administration (P<0.05) with a maximal decrease observed after 120 min (P<0.005). Furthermore, oral administration of peptide 3 resulted in a more profound decrease in blood glucose at 120 min, compared to intraperitoneal administration of peptide 3 at the same time point (P<0.005).

### EXAMPLE 6

### Effect of Chronic Administration of Peptide 3

The effects of chronic administration of peptide 3 in the C57Bl/6J ob/ob diabetic model were evaluated by measurements of a number of parameters, including body weight, food intake, plasma glucose levels, plasma insulin levels, intraperitoneal glucose tolerance test and glucose uptake by adipose tissue (ex vivo).

Male and female C57BL/6J ob/ob mice aged 12-15 weeks old were used. Fasting blood glucose levels were determined for all mice 14 days prior to experimentation. Only mice with fasting blood glucose levels >7.0 mmol/l were used in the study.

Mice selected for this experiment were initially fasted for 4 hours, then anaesthetized with a single injection of sodium pentobarbitone (35 mg/kg). A blood sample was collected from each mouse by eye-bleed for the assessment of plasma glucose and insulin levels (day 0). Collected blood samples were stored at −20° C. until analysis was performed. A single Alzet mini-pump (#1002, Alzet, USA) containing either sterile saline (100 μl; n=14) or peptide 3 (20 μmol/kg dissolved in 100 μl saline; n=14) was inserted under the skin between the scapula of the mice. The incision was clamped and disinfected using iodine. The pumps were left for 14 days, and 5 body-weight, and food measurements were recorded during this period, at days 0, 4, 7, 10 and 14. Blood samples were collected on day 0 and 14 days post-saline (n=5) or peptide 3 (n=4) administration for plasma glucose analysis. The results reported below are expresses as mean +/− standard error.

(a) Plasma insulin Plasma insulin levels were quantitatively determined in ob/ob mice, using an insulin radioimmunoassay kit (Linco Research Inc. USA) according to the manufacturer's instructions. Plasma insulin was quantitated for saline-treated (n=4) and peptide 3-treated mice (n=4).

(b) Intraperitoneal glucose tolerance test (IPGTT)

An intraperitoneal glucose tolerance test was conducted to determine whether the clearance of a glucose load was enhanced. Ten mice were used in each group, five receiving saline and five receiving peptide 3. At 14 days after chronic administration of saline or peptide 3, mice were fasted for 4 hours, then anaesthetized and eye-bled for day 14 plasma metabolite analysis. Half of each saline or peptide 3 treatment group was given a single intraperitoneal injection of glucose (1 g/kg dissolved in saline), and the other half saline (equivalent dose). Mice were eye-bled at 30, 60 and 120 minutes after glucose administration, and blood glucose levels were determined.

(c) Glucose transport assay Glucose transport in adipose tissue extracted from saline-treated and chronically peptide 3-treated mice was analyzed by an ex vivo glucose uptake assay. Adipose tissues was harvested from mice which had received saline (n=5) or peptide 3 (n=4) in the IPGTT, and used for a glucose transport assay. Mice were sacrificed by a lethal injection of pentabarbitone to the heart. Epididymal fat from male mice or peritoneal fat from female mice was used. Adipose tissues were rinsed in saline and then sliced into even pieces for weight determination. Tissues were placed in flasks and incubated in 2 ml KRB buffer (pH 7.4) containing D-glucose (10 mM final concentration) with vigorous agitation at 37° C. for 2 h under an atmosphere of carbogen. All samples were then placed on ice to reduce glycolysis. Tissues were removed from flasks, and the remaining solutions were analyzed for glucose concentrations using a glucose analyzer. Glucose uptake by each tissue sample was calculated, and compared to tissue free buffer controls.

There was no significant difference in body weight gain or food intake between the saline-infused and peptide 3-infused mice over 14 days of treatment. However, plasma glucose levels were significantly decreased when mice were continuously infused with peptide 3 for 14 days, compared to saline-infused control mice (P<0.025). Mice treated with peptide 3 exhibited a reduction of 11.60±3.63 mmol/l in plasma glucose levels, compared to a negligible increase of 2.38±1.81 mmol/l in saline-infused mice (P<0.005), which is indicative of fasting (4h) plasma glucose measurements. These data suggest that chronic peptide 3 treatment significantly improved glucose clearance from the circulation.

The plasma insulin level observed in mice treated with peptide 3 for 14 days was significantly reduced compared to saline-treated mice (17.10±5.99 ng/ml and 52.75±10.10 ng/ml respectively; P<0.01). This suggests that mice chronically treated with peptide 3 produce less insulin, as their blood glucose is being cleared more efficiently from the circulation and glucose transport into specific tissues such as adipose tissue is increased, as demonstrated in this study (see below).

Prior to glucose injection, peptide 3-treated mice were demonstrated to have a lower basal blood glucose level of 46.1% compared to saline-treated mice (P<0.01). The injection of a bolus of glucose into mice resulted in an increase in plasma glucose by 115% in peptide 3-treated and saline-treated mice respectively after 30 min. However, the level of blood glucose in peptide 3-treated mice was reduced by 47% at 120 min after glucose injection compared to saline-treated mice; this decrease was significant (P<0.03). These results suggest that glucose is cleared more efficiently in mice chronically treated with peptide 3, and therefore a reduced hyperglycaemic effect is observed following glucose load.

Adipose tissue extracted from mice treated with peptide 3 for 14 days was shown to transport 38% more glucose (1.67±0.18 nmol/mg tissue/h) than adipose tissue from saline-treated mice (1.22±0.18 nmol/mg tissue/h (P<0.05). Thus chronic administration of peptide 3 results in enhanced glucose removal from the circulation to tissue, where it may be stored as fat or oxidized for energy utilization.

### EXAMPLE 7

### Analysis of Synthetic Peptide Analogues

In this example the peptide analogues were manually synthesized using solid-phase peptide synthesis by the Fmoc-strategy on a Rink amide acid, DIC (diisopropylcarbodiimide) and HOBt (1-hydroxybenzotriazol), using conditions slightly modified from those described above. Coupling was complete after incubation for 2 h. Fmoc was removed with piperidine/DMF. The final peptides were cleaved from the resin by treatment with trifluoroacetic acid, crystalline phenol, EDT and thioanisole. The filtrate from the cleavage reaction was precipitated in the ether solvent at 0° C. The precipitate was dissolved in acetonitrile/H₂O.

Peptides were purified by reversed-phase high performance liquid chromatography using a preparative C18 column and an acetonitrile gradient.

The activity of each analogue was assessed by in vitro measurement of glycogen synthesis in muscle, as described above.

The amino acids tested for each position in the tetrapeptide of general formula W-X-Y-Z as defined in the “Summary of the Invention” are set out in Table 4, and the activity results are summarized in Tables 5 and 6.

TABLE 4

| W | X | Y | Z |
|---|---|---|---|
| Lysine | Glycine | Phenylalanine | Phenylalanine |
| Arginine | Leucine | Tyrosine | Tyrosine |
| Homolysine | Alanine | | Tryptophan |
| Homoarginine | β-Alanine | | Histidine |
| Ornithine | Isoleucine | | Homophenylanine |
| | Cyclohexylalanine | | |

TABLE 5
Sequences of synthetic peptides and activity as measured in an in vitro
glycogen synthesis assay in Zucker (fa/fa) rat hemidiaphragm muscle.

| PEP- | | PEPTIDE AMIDE | |
|---|---|---|---|
| TIDE | SEQUENCE | ANALOGUE | ACTIVITY |
| 1 | INSB (22-25) | Arg-Gly-Phe-Phe | insulin-poten- |
| | | | tiating * |
| 2 | Cha²⁵-INSB (22-25) | Arg-Gly-Phe-Cha | insulin antago- |
| | | | nist |
| 3 | D-Ala23-INSB (22-25) | Arg-D-Ala-Phe-Phe | insulin-poten- |
| | | | tiating ** |
| 4 | INSREC (86-89) | Arg-Leu-Phe-Phe | insulin-poten- |
| | | | tiating * |
| 5 | hGH (8-13) | Arg-Leu-Phe-Asp- | inactive |
| | | Asn-Ala | |
| 6 | Asu¹¹-hGH (8-13) | Arg-Leu-Phe-Asu- | insulin-poten- |
| | | Asn-Ala | tiating * |
| 7 | Asu¹¹-hGH (6-13) | Leu-Ser-Arg-Leu- | insulin-poten- |
| | | Phe-Asu-Asn-Ala | tiating * |
| 8 | Lys⁸, Asu¹¹-hGH (6-13) | Leu-Ser-Lys-Leu- | insulin-poten- |
| | | Phe-Asu-Asn-Ala | tiating * |
| 9 | Gly⁸, Asu¹¹-hGH (6-13) | Leu-Ser-Gly-Leu- | inactive |
| | | Phe-Asu-Asn-Ala | |
| 10 | Tyr¹⁰, Asu¹¹-hGH (6-13) | Leu-Ser-Arg-Leu- | insulin-poten- |
| | | Tyr-Asu-Asn-Ala | tiating * |
| 11 | Gly¹⁰, Asu¹¹-hGH (6-13) | Leu-Ser-Arg-Leu- | inactive |
| | | Gly-Asu-Asn-Ala | |
| 12 | Ala²²-INSB (22-25) | Ala-Gly-Phe-Phe | inactive |
| 13 | Ala²³-INSB (22-25) | Arg-Ala-Phe-Phe | inactive |
| 14 | Ala²⁴-INSB (22-25) | Arg-Gly-Ala-Phe | inactive |
| 15 | Ala²⁵-INSB (22-25) | Arg-Gly-Phe-Ala | inactive |
| 16 | Lys²²-INSB (22-25) | Lys-Gly-Phe-Phe | inactive |
| 17 | Orn²²-INSB (22-25) | Orn-Gly-Phe-Phe | inactive |
| 18 | β-Ala²³-TNSB (22-25) | Arg-β-Ala-Phe-Phe | insulin-poten- |
| | | | tiating * |
| 19 | Tyr²⁴-INSB (22-25) | Arg-Gly-Tyr-Phe | insulin-poten- |
| | | | tiating * |
| 20 | Cha²⁴-INSB (22-25) | Arg-Gly-Cha-Phe | insulin antago- |
| | | | nist |
| 21 | Tyr²⁵-INSB (22-25) | Arg-Gly-Phe-Tyr | inactive |
| 22 | D-Ala²³-INSB (22-25) | Arg-D-Ala-Phe-Tyr | insulin-poten- |
| | | | tiating ** |
| 23 | D-Ala²³, Tyr-me²⁵-INSB | Arg-D-Ala-Phe-Tyr- | insulin-poten- |
| | (22-25) | me | tiating ** |
| 24 | D-Ala²³, Tyr²⁴-INSB (22- | Arg-D-Ala-Tyr-Phe | insulin-poten- |
| | 25) | | tiating *** |

INSB: insulin B-chain
INSREC: α-subunit of the insulin receptor
hGH: human growth hormone
Asu: aminosuccinimide
Cha: β-cyclohexyl-L-alanine
Orn: ornithine
All residues are of L-configuration unless indicated by “D”.
Activity:
insulin-potentiating: improved activity compared to insulin alone
inactive: equal activity compared to insulin alone
insulin antagonist: reduced activity compared to insulin alone
*-** increasing insulin-potentiating activity

The following conclusions regarding the activity of the INSB tetrapeptides can be drawn from the results presented in Table 5, and are summarised in Table 6:

Position W: Arginine seems to be required for activity for the INSB tetrapeptides. When lysine (peptide 16) or ornithine (peptide 17) is substituted for arginine there is a loss of activity.

Position X: All possible substitutions have not yet been tested in this position. However, for glycine the activity seems to be determined by the amino acids that follow, ie. positions Y and Position, Z: Alanine is inactive, but the D-alanine and β-alanine forms are active.

Position Y: Phenylalanine and tyrosine can be replaced, but activity is determined by the amino acid preceding this position ie. amino acid X.

Position Z: Only phenylalanine and tyrosine have been tested in this position. Again, activity is determined by the amino acid in position X.

However, the activity of longer peptides may be modulated by the N- or C-terminal extension; for example, peptide 8 is active, although it has lysine instead of arginine at position W.

The amino acid substitutions of the tetrapeptide allow the aromatic rings and side chains to maintain a conformation that allows high affinity binding to the target sequence.

TABLE 6
(corrected)

| Peptide | | |
|---|---|---|
| (SEQ ID NO) | Sequence | Activity |
| 1 | Arg-Gly-Phe-Phe-NH₂ | active |
| 2 | Arg-Gly-Phe-Cha-NH₂ | antagonist |
| 14 | Arg-Gly-Ala-Phe-NH₂ | inactive |
| 15 | Arg-Gly-Phe-Ala-NH₂ | inactive |
| 19 | Arg-Gly-Tyr-Phe-NH₂ | active |
| 20 | Arg-Gly-Cha-Phe-NH₂ | antagonist |
| 21 | Arg-Gly-Phe-Tyr-NH₂ | inactive |
| 4 | Arg-Leu-Phe-Phe-NH₂ | active |
| 13 | Arg-Ala-Phe-Phe-NH₂ | inactive |
| 3 | Arg-D-Ala-Phe-Phe-NH₂ | active |
| 18 | Arg-β-A1a-Phe-Phe-NH₂ | active |
| 22 | Arg-D-Ala-Phe-Tyr-NH₂ | active |
| 23 | Arg-D-Ala-Phe-Tyr-me-NH₂ | active |
| 24 | Arg-D-Ala-Tyr-Phe-NH₂ | active |

### DISCUSSION

The insulin-potentiating effect of INSB(22-25)-NH₂, a tetrapeptide amide, has been demonstrated in normal rats (Ng et al, 1989; Weitzel et al, 1971). The evidence indicated that the amino acid sequence is essential for hormonal function. The Arg^{B22} residue is important for bioactivity, since an Ala^{B22}-substituted analogue was found to be inactive (Weitzel et al, 1971). The guanidinium functional group of Arg frequently plays a crucial role in the biological activities of proteins and peptides (Hannon and Anslyn, 1993). Phe^{B24} and Phe^{B25} are two residues which are invariant and important in animal insulins during evolution, and are critical for receptor binding. Tager et al (1979) reported the discovery of a mutant insulin from a diabetic patient in which the phenylalanine at B24 or B25 is replaced by leucine, and showed that the activity of the mutant insulin was reduced almost one hundred fold. It has been suggested that the Phe^{B25} residue of the insulin molecule interacts with the Phe⁸⁹ of the α-subunit of the insulin receptor molecule by means of an aromatic-aromatic interaction, resulting in hormone-receptor binding (Sabesan and Harper, 1980).

In the present study, the insulin-potentiating effects of peptide analogues derived from insulin, insulin receptor and hGH were examined both in vitro and in vivo. Peptide analogues were designed and synthesized in order to identify those residues responsible for bioactivity (Tables 2, 5 and 6). Our results indicated that the Arg-Gly-Phe-Phe (i.e. INSB(22-25)) amide peptide had insulin-potentiating effects; it stimulated glycogen synthesis in tissues in vitro, and reduced basal blood glucose levels in vivo in insulin-resistant Zucker fatty (fa/fa) rats. The findings with the INSB(22-25) peptide are consistent with our previous observation of a similar effect during IVITT in normal Wistar rats (Ng et al, 1989).

An increased insulin-potentiating effect was observed when Gly^{B23} was replaced by a D-Ala residue. In particular, significantly increased (p<0.05) on in vitro glycogen synthesis was observed in the presence of 0.01-1 μmol/ml Arg-D-Ala-Phe-Phe amide (FIG. 2A). Increased potency of the in vivo hypoglycaemic effect of this D-Ala substituted peptide analogue was also observed (FIG. 3). This change is likely to prevent the degradation of D-Ala^{B23}-INSB(22-25) by the proteolytic attack of tissue enzymes, as is usually observed in peptides with D-amino acid substitutions (Zhang, 1989). INSREC(86-89) amide displayed similar but less striking effects both in vitro and in vivo.

In contrast, the bioactivity was lost when the Phe^{B25} residue was substituted by its saturated counterpart, β-cyclohexyl-L-alanine(Cha) (Armstrong et al, 1993 and FIGS. 2A & 3). Asu¹¹-hGH(8-13) amide, in which residues 8-11 are homologous to INSREC(86-89), showed a diminished insulin-potentiating effect (FIG. 2A and FIG. 3). The Asu¹¹ group may mimic the molecular structure of the aromatic side-chain of the Phe^{B25} residue. However, the decrease in activity may result from facile hydrolytic opening of the α-aminosuccinimide ring at physiological temperature and pH (Table 2). Our evidence suggests that the residue at this position of the tetrapeptide motif should be of an unsaturated and cyclic structure to elicit the insulin-potentiating effect.

The insulin-potentiating effects of the peptides were further confirmed by results of intravenous insulin tolerance tests (IVITTs) with a series of hGH peptide analogues. Structure-activity relationships of peptide analogues revealed that the Arg⁸, Phe¹⁰and Asu¹¹ residues are crucial for bioactivity. Replacement of Arg⁸ or Phe¹⁰ with Lys or Tyr respectively showed equivalent insulin-potentiating activity because of the structural similarity between Arg and Lys and between Phe and Tyr. The activity was dramatically reduced when residue 8 or 10 was substituted by Gly (Tables 3,5). Asu¹¹-hGH(8-13) peptide amide showed a similar but less potent bioactivity than that of Asu¹¹-hGH(6-13) peptide amide (Tables 3,5). However, linear hGH(8-13) had no activity. Robson also showed that the bioactivity of hGH peptides was lost when the Asu residue was substituted by an acyclic amino acid such as Ala, Asp or Gly (Robson, 1986).

In summary, our results clearly indicate that the insulin-potentiating activity is characteristic of a molecular motif with sequence homology to amino acid residues 22-25 of the B-chain of insulin, residues 86-89 of the α-subunit of insulin receptor and residues 8-11 of hGH. This biological activity appears to be intrinsic to a four-residue motif with a basic amino acid in position 1, a neutral aliphatic amino acid in position 2, an aromatic amino acid in position 3, and an amino acid with a side-chain having π or non-binding electrons in position 4. The insulin-potentiating effect of Asu¹¹-hGH(6-13) peptide has been shown to be mediated by stimulating insulin receptor tyrosine kinase activity (Lim et al, 1994).

It will be apparent to the person skilled in the art that while the invention has been described in some detail for the purposes of clarity and understanding, various modifications and alterations to the embodiments and methods described herein may be made without departing from the scope of the inventive concept disclosed in this specification.

References cited herein are listed on the following pages, and are incorporated herein by this reference.

### REFERENCES

Armstrong, K. M., Fairman, R. and Baldwin, R. L. J. Mol. Biol., 1993 230 284-291

Baba, T. and S. Neugebauer Drugs, 1994 47 383-404

Chorev and Goodman Acc. Chem. Res., 1993 26 266-273

De Meyts, P., Gu, J. L., Shymko, R. M., Kaplan, B. E., Bell, G. I. and Whittaker, J. Mol. Endo., 1990 4 409-416

Ede, N. J., Rae, I. D. and Hearn, M. T. W. Int. J. Pept. Prot. Res., 1994 44 568-581

Felber, J. P., Acheson, K. J. and L. Tappy, From Obesity to Diabetes, J. P. Felber, K. J. Acheson and L. Tappy (Eds), 123-140, John Wiley & Sons Ltd., England (1993)

Gallop, M. A., Barrett, R. W., Dower, W. J., Fodor, S. P. A. and Gordon, E. M. J. Med. Chem., 1994 37 1233-1251

Hannon, C. L. and Anslyn, E. V. Bioorganic chemistry Frontiers, H. Dugas and F. P. Schmidtchen (Eds), 1993 193-255, Springer-Verlag, Berlin

Hogan, Jr., J. C. Nature Biotechnology, 1997 15 328-330

Keen, H. N. Engl. J. Med., 1994 331 1226-1227

King, D. S., Fields, C. G. and Fields, G. B. Int. J. Pept Prot. Res., 1990 36 255-266

Lim, N., Jiang, W. J., Graham, L. and Ng, F. M. Proceeding in Annual Scientific Meeting of Australian Diabetes Society, 111 (Poster 64) (1995).

Lim, N., Ng, F. M. and Hearn, M. T. W. Endocrinology, 1992 131 835-840

Lim, N., Wijaya, E. and Ng, F. M. Life Sciences, 1994 54 1471-1481

Ng, F. M., Bornstein, J., Pullin, C. E., Bromley, J. O. and Macaulay, S. L. Diabetes, 1980 29 782-787

Ng, F. M., Zhu, S. Q., Cui, D. F., Fan, L., Huang, Y. D. and Zhang, Y. S. Biochem. Mol. Biol. Int., 1989 18 373-381

Olson et al J. Med. Chem., 1993 36 3039-3049

Pullen, R. A., Lindsay, D. G., Wood, S. P., Tickle, I. J., Blundell, T. L., Wollmer, A., Krail, G., Brandenburg, D., Gliemann, J. and Gammeltoft, S. Nature 1976 259 369-373

Robson, V. M. J. Ph. D. Thesis, Monash University, Victoria, Australia, 1986

Robson, V. M. J. and Ng, F. M. Biol. Chem. Hoppe. Seyler, 1990 371 423-341

Sabesan, M. N. and Harper, E. T. J. Theor. Biol., 1980 83 457-467

Stephenson, R. C. and Clarke, S. J. Biol. Chem., 1989 264 6164-6170

Tager, H., Given, B., Baldwin, D., Mako, M., Markese, J., Rubenstein, A., Olefsky, J., Kobayashi, M., Kolterman, O. and Poucher, R. Nature 1979 281 122-125

Truglia, J. A., Livingston, D. V. M. and Lockwood, D. H. Am. J. Med., 1985 979 13-22.

Weitzel, G., Weber, U., Martin, J. and Eisele, K. Hoppe. Seyler's Z. Physiol. Chem., 1971 354 321-330

Zavaroni, I., Bonora, E., Pagliara, M., Dallaglio, E., Luchetti, L., Buonanno, G., Bonati, P. A., Bergonzani, M., Gnudi, L., Passeri, M. and Reaven, G. N. Engl. J. Med., 1989 320 702-706

Zhang, Y. S. Current Biochemical Research in China, C. L. Tsou (Eds), 1989 22 129-136, Academic Press, Inc., California

The Shanghai Insulin Research Group Sci. Sin. 1974 17 779-787

1. A peptide or peptidomimetic compound which has the ability to potentiate one or more of the physiological activities of insulin, in which the peptide comprises the sequence:
W-X-Y-Z
in which W is a basic amino acid;
X is a neutral aliphatic amino acid, in either the L- or the D-form;
Y is an aromatic amino acid; and
Z is an amino acid or amino acid analogue which has a side chain having π or delocalised electrons,
with the proviso that where the compound is a peptide, it is not Arg-Gly-Phe-Phe, Arg-Gly-Ser-Arg-Leu-Phe-Phe-Asn-Tyr-Ala-Leu-Val, Arg-Leu-Phe-Asu-Asn-Ala, or Leu-Ser-Arg-Leu-Phe-Asu-Asn-Ala.

2. A peptide according to claim 1, in which
W is lysine, arginine, homolysine, homoarginine or ornithine;
X is the L- or the D-form of glycine, leucine, alanine, β-alanine, isoleucine, homoleucine, norleucine, homonorleucine, cyclohexylalanine, or homocyclohexylalanine; and/or
Y is phenylalanine or tyrosine.

3. A peptide according to claim 1 or claim 2, in which the amino acid or amino acid analogue Z is one with a cyclic side chain.

4. A peptide according to any one of claims 1 to 3, in which Z is phenylalanine, tyrosine, tryptophan, α-amino succinimide, homophenylalanine or histidine.

5. A peptide according to any one of claims 1 to 4, in which W is arginine.

6. A peptide according to any one of claims 1 to 5, in which X is glycine, D-alanine, or β-alanine.

7. A peptide according to any one of claims 1 to 5, in which Y is phenylalanine or tyrosine.

8. A peptide according to any one of claims 1 to 7, in which Z is phenylalanine, tyrosine, or methyl-tyrosine.

9. A peptide according to any one of claims 1 to 8, which is extended at either the N- or C-terminal.

10. A peptide according to claim 9, in which the N-terminal extension is leucine-serine.

11. A peptide according to claim 9, in which the C-terminal extension is asparagine-alanine.

12. A peptide according to any one of claims 1 to 11, selected from the group consisting of

| | |
|---|---|
| Arg-D-Ala-Phe-Phe, | (SEQ ID NO. 3) |
| Arg-Leu-Phe-Phe, | (SEQ ID NO. 4) |
| Arg-Leu-Phe-Asu-Asn-Ala, | (SEQ ID NO. 6) |
| Leu-Ser-Arg-Leu-Tyr-Asu-Asn-Ala, | (SEQ ID NO. 7) |
| Leu-Ser-Lys-Leu-Phe-Asu-Asn-Ala, | (SEQ ID NO. 8) |
| Leu-Ser-Arg-Leu-Tyr-Asu-Asn-A1a, | (SEQ ID NO. 10) |
| Arg-β-Ala-Phe-Phe, | (SEQ ID NO. 18) |
| Arg-Gly-Tyr-Phe, | (SEQ ID NO. 19) |
| Arg-D-Ala-Phe-Tyr, | (SEQ ID NO. 22) |
| Arg-D-Ala-Phe-Tyr-me, and | (SEQ ID NO. 23) |
| Arg-D-Ala-Tyr-Phe. | (SEQ ID NO. 24) |

13. A peptide according to claim 12, which is Arg-D-Ala-Phe-Phe (SEQ ID NO. 3) or Arg-D-Ala-Tyr-Phe (SEQ ID NO. 24).

14. A peptidomimetic compound according to claim 1, in which W is replaced by an analogue of arginine.

15. A peptidomimetic compound according to claim 1 or claim 14, in which
(a) one or more amino acids is replaced by its corresponding D-amino acid, or
(b) one or more peptide bonds is replaced by a structure more resistant to metabolic degradation.

16. A composition comprising a peptide according to any one of claims 1 to 13, or a peptidomimetic compound according to claim 14 or claim 15, together with a pharmaceutically-acceptable carrier.

17. A method of treatment of a pathological condition associated with insulin resistance, comprising the step of administering an effective amount of a peptide according to according to any one of claims 1 to 13, or a peptidomimetic compound according to claim 14 or claim 15, to a subject in need of such treatment.

18. A method according to claim 17, in which the condition is non-insulin dependent diabetes mellitus or obesity.

19. A method according to claim 17 or claim 18, in which the condition is non-insulin dependent diabetes mellitus.

20. A method according to any one of claims 17 to 19, in which the peptide or peptidomimetic compound is administered at a dose in the range 0.1 to 100 mg/kg body weight.

21. A method according to any one of claims 17 to 20, in which the peptide or peptidomimetic compound is administered orally or sublingually.

22. A method of treatment of a pathological condition associated with insulin resistance, comprising the step of administering an effective amount of a compound which mimics the action of the binding region INSB 22:25 on the insulin receptor.

23. A method according to claim 23, in which the condition is non-insulin dependent diabetes mellitus.

24. Use of a peptide according to according to any one of claims 1 to 13, or a peptidomimetic compound according to claim 14 or claim 15, for the manufacture of a medicament for the treatment of a pathological condition associated with insulin resistance.

25. Use according to claim 23, in which the condition is non-insulin dependent diabetes mellitus or obesity.

26. Use according to claim 23 or claim 24, in which the condition is non-insulin dependent diabetes mellitus.

27. Use according to any one of claims 23 to 25, in which the peptide or peptidomimetic compound is administered at a dose in the range 0.1 to 100 mg/kg body weight.

28. Use according to any one of claims 23 to 26, in which the peptide or peptidomimetic compound is administered orally or sublingually.
